(19) United States
(12) Patent Application Publication
(10) Pub. No.: US 2015/0313578 A1
(43) Pub. Date: Nov. 5, 2015

(54) Multi-user wireless ulttrasound server

(71) Applicant: Siemens Medical Solutions USA, Inc., Malvern, PA (US)

(72) Inventors: Daphne Yu, Yardley, PA (US); Ankur Kapoor, Plainsboro, NJ (US); Christophe Chefd'hotel, Jersey City, NJ (US); Peter Mountney, Princeton, NJ (US); Mamadou Diallo, Plainsboro, NJ (US); Dorin Comaniciu, Princeton, NJ (US); Gianluca Paladini, Skillman, NJ (US)

(73) Assignee: Siemens Medical Solutions USA, Inc., Malvern, PA (US)

(21) Appl. No.: 14/270,118

(22) Filed: May 5, 2014

(51) Int. Cl. A61B 8/00; A61B 8/08

(52) U.S. Cl. CPC A61B 8/565; A61B 8/5207; A61B 8/466; A61B 8/4254; A61B 8/523; A61B 8/4472; A61B 8/4427

(57) ABSTRACT

Multiple users are supported with an ultrasound server. Tiling of images may be used to limit transmission and/or bandwidth. By transmitting parts of images that change and avoiding transmission of other parts, wireless and processing bandwidth may be optimized. On the server side, separate instances are used for scanning each patient or for each of the multiple transducer probes being used. Dynamic assignment of shared resources based on use of the transducer probes may provide further optimization. From an overall perspective, the server may beamform from data received by a transducer probe based on controls routed from a separate tablet used as a display and user input.

### BACKGROUND

The present embodiments relate to ultrasound imaging. Traditional ultrasound systems include a computer and an attached display on a wheeled trolley with a connected transducer. The trolley form factor is often cumbersome due to limited space and may require the sonographer to reach around the patient to operate the ultrasound system. Portable systems reduce the large computer system and display to laptop or smaller portable sizes, but the computational power and small display limits the image quality and analytic features available. More recent wireless transducer technology further allows for detached operation between the transducer and the computer and display, thus greatly improving the reach to the patient. Nevertheless, the operator is still required to be within close vicinity of the display to view the imaging results, thus also binding the operator and patient very close to a supporting computer system. The computers of wireless ultrasound systems are limited in size and computational power due to the need to bring the ultrasound system close to patient bed side or typical small imaging rooms.

Multi-transducer portable ultrasound systems have been proposed using a shared image processing resource. However, the simple approach to this arrangement may not be practical in terms of cost and operation. Transmission and computing resources may be a constraint. Single user wireless ultrasound system and probe design may overcome these concerns by avoiding multi-user complexities, but may be inefficient or provide only limited image processing.

### BRIEF SUMMARY

The present invention is defined by the following claims, and nothing in this section should be taken as a limitation on those claims. By way of introduction, the preferred embodiments described below include methods, computer readable media, instructions, and systems for supporting multiple users with an ultrasound server. Tiling and layering of images may be used to limit transmission and/or bandwidth. By transmitting parts of images that change and avoiding transmission of other parts, wireless and processing bandwidth may be optimized. On the server side, separate instances are used for scanning each patient or for each of the multiple transducer probes being used. Dynamic assignment of shared resources based on use of the transducer probes may provide further optimization. From an overall perspective, the server may beamform from data received by a transducer probe based on controls routed from a separate tablet used as a display and user input. Any one or combination of multiple of these approaches may be used to realize practical and cost efficient multi-transducer probe, server-based ultrasound imaging.

In a first aspect, a method is provided for supporting multiple users with an ultrasound server. A local area server receives ultrasound scan data from a handheld transducer probe. The local area server generates an ultrasound image representing a rendering from the data. The ultrasound image is formed as a plurality of tiles. The local area server transmits the ultrasound image to a display. A change for the rendering is received. A first sub-set of the tiles of the ultrasound image that are different due to the change and a second sub-set of the tiles that are not different are determined. The local area server renders the tiles of the first sub-set. The rendered tiles of the first sub-set and not tiles of the second sub-set are transmitted to the display.

In a second aspect, a non-transitory computer readable storage medium has stored therein data representing instructions executable by a programmed processor for supporting multiple users with an ultrasound server. The storage medium includes instructions for communicating, wirelessly, with multiple ultrasound transducer probes, operating a separate instance of an image processing and control system for each of the ultrasound transducer probes, and image processing, for each of the image processing and control systems as part of the operating, data from the ultrasound transducer probes.

In a third aspect, a system is provided for supporting multiple users with ultrasound processing. A plurality of ultrasound probes are configured to scan patients and wirelessly output channel data. A plurality of tablet displays are paired with the ultrasound probes. Each of the tablet displays is configured to operate as a user input for control of the paired ultrasound probe. A server is configured to receive the channel data from the ultrasound probes, beamform the channel data, create images from the beamformed channel data as a function of the user input from the tablet displays, and transmit the images to the tablet displays, and configured to control the ultrasound probes as a function of the user input.

Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for supporting multiple users with ultrasound processing;

FIGS. 2 and 3 illustrate other embodiments of a system for supporting multiple users with ultrasound processing;

FIG. 4 is a function block diagram of one embodiment of a server with an ultrasound instance and a client with a transducer probe and display;

FIG. 5 is a flow chart diagram of one embodiment of a method for supporting multiple users with ultrasound processing;

FIGS. 6 and 7 show different embodiments of a server-client system for supporting multiple users with ultrasound processing;

FIG. 8 is a block diagram showing one embodiment of a system for sharing server resources amongst multiple ultrasound clients; and

FIG. 9 is a flow chart diagram of one embodiment of a method for operating and controlling an ultrasound application instance in supporting multiple users.

### DETAILED DESCRIPTION OF THE DRAWINGS AND PRESENTLY PREFERRED EMBODIMENTS

A multi-user wireless ultrasound server provides efficient ultrasound processing in a local area. A single (or multiple) ultrasound server supports multiple users simultaneously acquiring, viewing and analyzing ultrasound images. Each user potentially acquires different images and performs different tasks that require different computing and transmission resources of the server. Each user client may include a wireless ultrasound transducer coupled with a portable computing device (e.g., tablet computer) equipped with a high resolution display, a touch screen or other human input devices, and additional sensors, such as inertial sensors. The transducer and portable computing device communicate via wireless connection to the ultrasound server.

Such a multi-user system utilizes sensor information sent from ultrasound transducers and the portable computing devices, manages computing and multi-user resources, and provides optimal processing, viewing and rendering performance within limited wireless bandwidth and battery life. Techniques to optimally minimize rendering and interaction latency may utilize caching strategies on the portable computing device.

In order to maximize the number of concurrent users for each server while maintaining sufficient image quality for procedures and diagnostics, optimal use of image transmission bandwidth, optimal control of image quality, and optimal use of battery life may be provided. Exchange between the transducer, portable computing devices, and the server of transducer signals, sensor information, image streams and workflow context information may be optimized. Particularly when the amount of required client resources cannot be pre-determined, such as when the resource requirement is dependent on the user selected image acquisition and imaging tasks, dynamic server allocation based on sensor provided user activity information may provide significant server performance improvements over conservative static allocations. Sensor data provided by the client devices is used to drive the processing, rendering and transmission of images. Context aware software may improve image quality while minimizing bandwidth. In addition, scalable system designs may allow for dynamic use of computing resources based on dynamic resource needs.

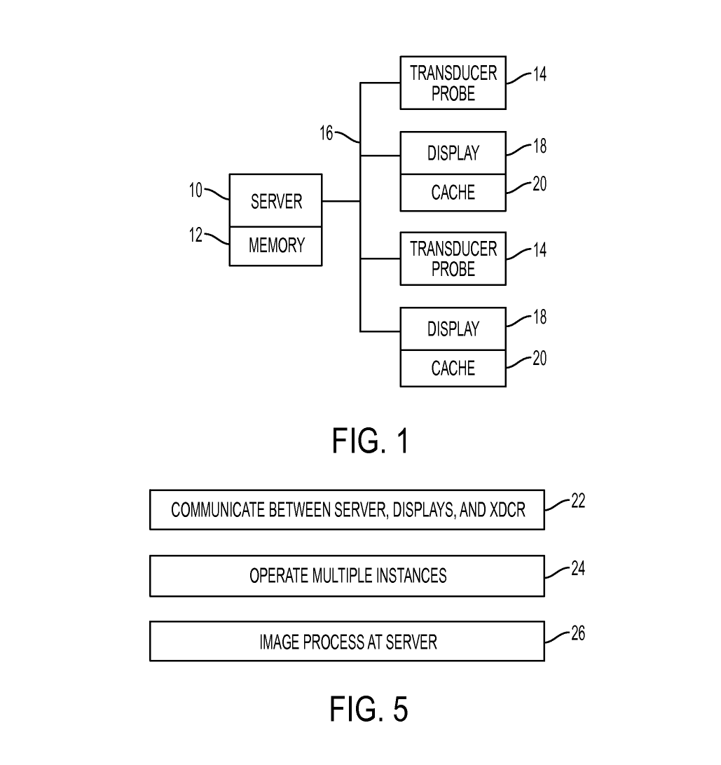

FIG. 1 shows a system for supporting multiple users with ultrasound processing. The system includes a server 10, a database or memory 12, client transducer probes 14, client displays 18, and a network 16. Additional, different or fewer components may be provided. For example, more than one server 10 is provided. The database 12 is shown local to the server 12, but may be remote from the server. More than one database 12 may be provided for interacting with any number of servers 10 exclusively or on a shared basis. Different networks may be used to connect different client transducer probes 14 and displays 18 to the server 10.

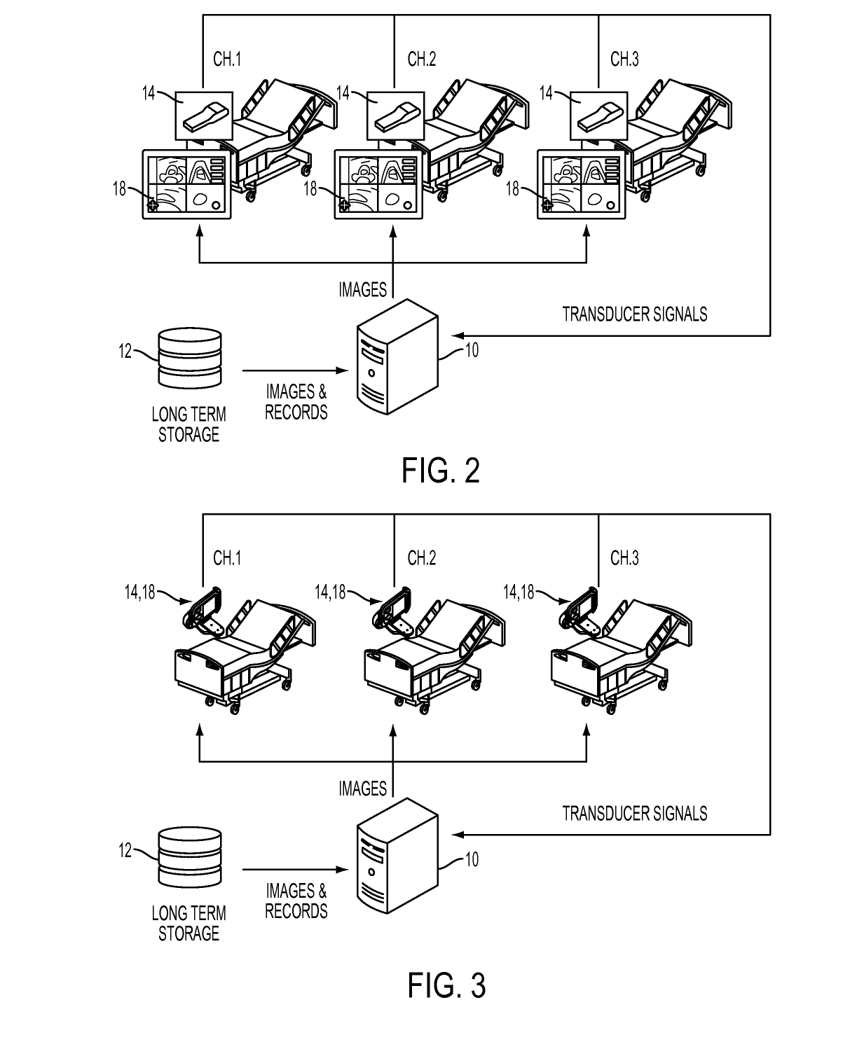

FIGS. 2 and 3 show examples of the system providing ultrasound imaging with a server 10 interacting with transducer probes 14 and displays 18 of multiple users. In FIG. 2, the transducer probes 14 and the displays 18 are separate devices, such as a handheld probe and a portable tablet (e.g., tablet computer). While treated as one client by the server 10, these separate devices are in different housings, have different power sources (e.g., batteries), and have different wireless interfaces. The server 10 may dedicate separate ports to these separate devices. In FIG. 3, the transducer probes 14 and displays 18 are combined into a single device with a single housing or with physically connected (e.g., cable) housings. The wireless transducer probe 14 includes a built-in or mounted on display 18. Where separate housings are connected by a cable, a same wireless interface and battery are used for both the transducer probe 14 and the display 18, but separate batteries may be provided.

In general, the ultrasound system is a server-based wireless ultrasound system where both the transducer probes 14 and the displays 18 are detached from the main computer system, thus allowing the operator to roam away from the heavy computer system to achieve fully flexible reach and use-ability. By detaching the transducer probes 14 and displays 18 from the computer processing unit, the computer no longer needs to have a small form factor to be practical or to interfere with scanning. This allows for the computer server 10 to support larger form factor and to support features that may require larger computational power, such as advanced image and business analytics. The server 10 may, in addition, be connected to other more permanently attached devices and other remote systems, such as through high-speed network access. For business analytics, the server 10 may have network connections and access to resources to assist in imaging. For example, the server 10 may connect with a picture archiving and communications system (PACS) as well as a patient information database. In addition, the server 10 may serve multiple transducer probes 14 and remote displays 18 for simultaneous operation with multiple patients within wireless range, such as multiple beds in a patient room or multiple rooms within a clinical office.

The transducer probes 14 generate acoustic energy for scanning patients, receive echoes, and wirelessly transmit the receive signals. In one embodiment, the transducer probe 14 includes a transmit beamformer, a transducer array, receive amplifiers, analog-to-digital converters, and a wireless transceiver. The transmit beamformer includes pulsers, a memory, timer, delays, phase adjustors, amplifiers, and/or other components for generating transmit beams of acoustic energy with electronic steering in an azimuth or azimuth and elevation directions. Using a phased, linear, curved linear, 1.5D or other array of 64, 128, 256 or other number of elements, the transmit beamformer causes generation of transmit beams along scan lines in a linear, sector, Vector® or other scan format.

For receive operation, the elements convert acoustic echoes into electrical signals as channel signals. The channel signals from the respective elements of the array are amplified with a time gain amplification level, digitized with the analog-to-digital converts, and wirelessly transmitted to the server 10 with the wireless transceiver. In other embodiments, the transducer probe 14 includes a receive beamformer or partial beamformer. Some or all of the channel data and/or signals from different channels are combined with relative delays or phasing and apodization.

The transducer probe 14 has a housing. The housing is sized or shaped to be handheld. For example, a single hand of a sonographer holds a grip on an outside of the housing. The housing encloses the rest of the transducer probe 14 so that the sonographer may move the transducer probe 14 around the patient with a single hand. In other embodiments, multiple housings are used, such as the sonographer wearing one part (e.g., transmit beamformer, battery, wireless transceiver, and other electronics) in a housing on a belt or held in one hand and holding in a hand another part (e.g., array) in another housing. The two housing are connected by a cable. In alternative embodiments, the electronics are in a laptop computer, briefcase-type device, or on a cart, connected by cable to the array in a handheld probe housing.

The transducer probe 14 includes a battery. The battery is rechargeable, such as using a charging station. Similarly, the display 18 includes a battery that is rechargeable, such as using the same or a different charging station. A plug or receptacle may be used for charging rather than a charging station. In alternative or additional embodiments, the transducer probe 14 and/or the display 18 are corded or physically plug into another source of power than a battery.

The transducer probe 14 and/or the display 18 also include one or more sensors, such as single or multi-touch input, video camera, gyroscope, accelerometers, buttons, dials, sliders, touch screens, touch pads, or other input device to provide usage feedback. For example, a touch input or pressure sensor is used to detect if the ultrasound transducer probe 14 is in contact with the patient skin and/or held by the sonographer. A combination of gyroscopes and accelerometers placed appropriately inside the ultrasound transducer probe casing may used to analyze transducer motion. As another example, the display 18 is a touch screen. Input signals, such as touch gestures (zoom, pan, rotate, slide, pinch), may be sent to the ultrasound server 10 to control the ultrasound imaging (e.g., scan and/or image processing parameters). As another example, the transducer probe 14 or display 18 may include a microphone, camera, or other sensor for detecting human inputs other than touch. Voice or hand/face gestures may be received and used to control the parameters of ultrasound. The sensor signals are sent to the server 10 where further analytics are performed to provide information overlay onto the display and/or to control the scan or image processing parameters of ultrasound imaging.

The display 18 is a liquid crystal diode (LCD) display, but may be a projector or other type of display. The display 18 is a computing device, such as a tablet computer, laptop computer, personal computer, or workstation with an output for presenting images. In one embodiment, the display 18 is portable, such as a tablet computer. In other embodiments, the display 18 is fixed, cart mounted, or of sufficient size and/or weight to remain stationary. The display 18 includes a housing, cache 20, battery, wireless transceiver, and/or other electronics. Additional, different, or fewer components may be provided.

The display 18 includes an operating system and application or a program for displaying ultrasound images received from the server 10. Ultrasound images received wirelessly from the server 10 are directly displayed. Alternatively or additionally, the images may be cached in the cache 20. Image processing, such as filtering or adding graphics, may occur in the display 18 to alter the image to be displayed.

The display 18 includes an application or program for providing a user interface with the sonographer. Control functions for the ultrasound scanning may be manipulated by the sonographer on the display 18. For example, buttons, sliders, dials, menus, input boxes, or other touch screen user interface options are displayed to the user for configuring the server 10, transducer probe 14, and/or display 18 for generating and displaying ultrasound images of a patient. Other sensors may be provided, such as a camera, microphone, gyroscope, and/or accelerometers, for controlling ultrasound imaging.

Each display 18 is paired with a respective transducer probe 14. The pairing is fixed or dynamic. For fixed, the display 18 is coded to communicate with the server 10 for interaction with the paired transducer probe 14, and vice versa. Alternatively or additionally, the display 18 and transducer probe 14 communicate directly without passing through the server 10. For dynamic pairing, the code is programmable. Using user input, timing relative to powering on, assignment by the server 10, and/or relative location (e.g., in the same room), the transducer probe 14 and display 18 are paired.

The cache 20 is a memory, such as graphics device memory, a random access memory, shared graphics and main memory, solid state drive, hard drive, or other type of memory. The cache 20 stores image information. For example, the cache 20 implements a CINE memory, storing a sequence of images in a first-in first-out or loop format. The cache 20 may be used to output images without receiving images from the server in a playback operation (e.g., rewind or display again recently displayed images).

In one embodiment, the cache 20 stores the image information as tiles. For example, a given image (e.g., 512×512) is stored as 9, 16, 25, 36, 49, 100, 144 or other number of separate regions. The tile regions do not overlap, but may overlap. The display 18 includes a processor, such as graphics processing unit (GPU) or a central processing unit (CPU), that assembles one or more images from the tiles. Similar to caching full images, the tiling operation allows reuse of tiles from previous images in a current image. Where only some tiles change between two images, the tiles that do not change are not re-transmitted by the server 10. Instead, those tiles stored in the cache 20 are reused for the subsequent image, reducing bandwidth.

The caching of images and/or tiles may be specific to the type of scan mode. For example, one layer for B-mode is provided with caching. Another layer for Doppler or flow mode is provided with separate caching. The display 18 may assemble an image to be displayed from the different layers. The contribution for each layer is assembled from the tiles for that layer. In alternative embodiments, the caching is of the combined imaging mode (e.g., cache images and/or tiles for a combined B-mode, Doppler image).

The display 18, such as with the GPU and/or CPU, performs little image processing, such as user interface and image assembly processing. Alternatively, the display 18 performs spatial or temporal filtering processes. Contrast, brightness, depth gain, gain, or other operations may be performed by the display 18. Other image processing may be provided, such as performing some aspects of volume rendering.

The server 10 performs image processing for generating the ultrasound image to be displayed on the display 18. The data received from the transducer probe 14 is used to generate the ultrasound image.

The server 10 includes one or more processors and is a workstation, part of a server bank, or an individual server. The server 10 includes ports for communicating through the network 16 with the transducer probe 14 and/or display 18. The ports are part of a wireless interface. The server 10 interacts with a set of clients to render and stream the data to or from those clients for ultrasound visualization. Ultrasound data is processed to provide an acceptable visual representation for the displays 18. Data from different transducer probes 14 are used to generate different images for respective different displays 18. The server 10 may continuously stream image data depending on the type of connected client while multiplexing requests from multiple clients to provide different images.

The transducer probe 14 and/or display 18 are homogeneous or heterogeneous in terms of client capabilities. For example, different amounts of memory are available. Some may have embedded processors and others may instead or additionally have a graphic processing unit. The display capability may be different, such as the resolution and/or screen size. Images are requested from the server 10, received as a stream from the server 10, and presented to the user locally on the display 18. Using services provided by the server 10, the transducer probe 14 and/or display 18 interact with the server 10 by changing settings (e.g., changing viewpoint or other setting that modifies the current visualization).

The network 16 is a single network, such as a local area network. In one embodiment, the network 16 is a collection of dynamically established wireless links between the transducer probes 14 and displays 18 with the server 10. One or more relays may be provided. In other embodiments, indirect linking is used, such as wireless communications between a Wi-Fi access point and the transducer probe 14 and display 18 with wired linking from the Wi-Fi access point to the server 10. Alternatively, the network 16 is the Internet or a collection of networks. The communications links of the servers 10 and/or transducer probe 14 and/or display 18 may have the same or different capabilities. For example, some transducer probes 14 and/or displays 18 may connect through cellular communications (e.g., 3G) and others with LTE. Others may communicate using Bluetooth. Yet others may communicate using ultra wide band communications. The network 16 may have different capabilities for different connections. In one embodiment, the communications of all of the transducer probes 14 is through ultra wide band communications. The displays 18 communicate with ultra wide band, Bluetooth, and/or Wi-Fi.

In the system of FIG. 1, multiple clients may visualize data from a two-dimensional (2D) or three-dimensional (3D) region at the same time. There are two constraints that may potentially limit interactivity from the perspective of a client—1) the latency in a server rendering the requested image and 2) the latency in the transmission of that rendered image to the client. To provide the 3D rendering as near an experience to “local” as possible, techniques to adapt and/or reduce the use of communications channel and/or processing bandwidth may be provided.

The imaging is provided with interactivity. The client is agnostic to whether the data or hardware capabilities exist locally and may access high-end visualizations from lower power devices.

The imaging may be different for different situations. The server 10 may control the way the ultrasound data is used for imaging and streamed so that the available aggregate hardware and bandwidth are used to scale appropriately depending on the type of probes 14 and/or display 18 connected, the number of paired probes 14/displays 18 concurrently operating, and/or the type of operation for each pair.

In this server arrangement, the clients (e.g., transducer probes 14 and/or displays 18) communicate with the server 10. The server 10 includes ultrasound processing logic. The server ultrasound processing logic includes an image generation engine, corresponding graphics processing units, and a compression engine. Additional, different, or fewer components may be provided.

In the setup of FIG. 2 or 3, the clients interact with the user interfaces to request operation. The client machines request rendered content from “the cloud” or from the server 10. The server 10 streams images or other data to the client display in response to the request. The servicing of the content is transparent to the client.

The server 10 contains facilities for controlling the transducer probe 14 to scan a patient and transmit the data to the server 10, imaging from the ultrasound data, compressing the resulting image, streaming the resulting image to the display 18 in real-time, and processing incoming requests from the user input that may change the resulting image (i.e. change of viewpoint) or that change the processing. The server 10 acts as a repository and intelligent processor for ultrasound data, provides that ultrasound data to a client for visualization in the form of a set of images that change depending on client actions, makes decisions about the data based on the type of client connected (e.g., in the case of smaller form factor devices, a smaller image may be generated, saving on server time required to rasterize, compress, and transmit the resulting data), presents a service-oriented architecture that allows the client to request information about the ultrasound data (e.g. measurement, PMI, etc.), and provides for user control. Additional, different, or fewer actions may be provided.

These functions of the server 10 transform imaging and interaction into a service, moving the majority of the logic into the server 10 and using the client as a scanner (data acquisition) and presentation mechanism (e.g., display device) for images that are created and processed remotely. The server 10 manages and controls: each client connection, request, and current state (e.g. viewpoint, etc.). The server 10 may share resources between clients requesting the same data. Based on the type of client and the bandwidth available to the server and client, decisions about data quality of the image are made by the server to scale requests appropriately.

The server logic is responsible for accepting incoming connections from clients and retrieving the right data for a client. For three-dimensional imaging, the rendering engine is responsible for generating a rendered image based on the current data and viewpoint for each client. Graphical resources are managed by the rendering engine appropriately across multiple client connections. The rendering engine may dispatch work over multiple GPUs. The rendering engine applies coherence and acceleration algorithms (e.g. frame differencing, down sampling, tiling, etc.) to generate images, when appropriate.

The compression engine is responsible for generating a compressed representation of the image for each client and decompressing data received from the transducer probes 14. The compression engine schedules compression using CPU and/or GPU resources, as available.

Various distributions of ultrasound image processing may be provided between the transducer probe 14, server 10, and display 18. The distribution of processing may change over time, such as in response to processing bandwidth of the server and/or communications bandwidth. Alternatively, the distribution stays the same.

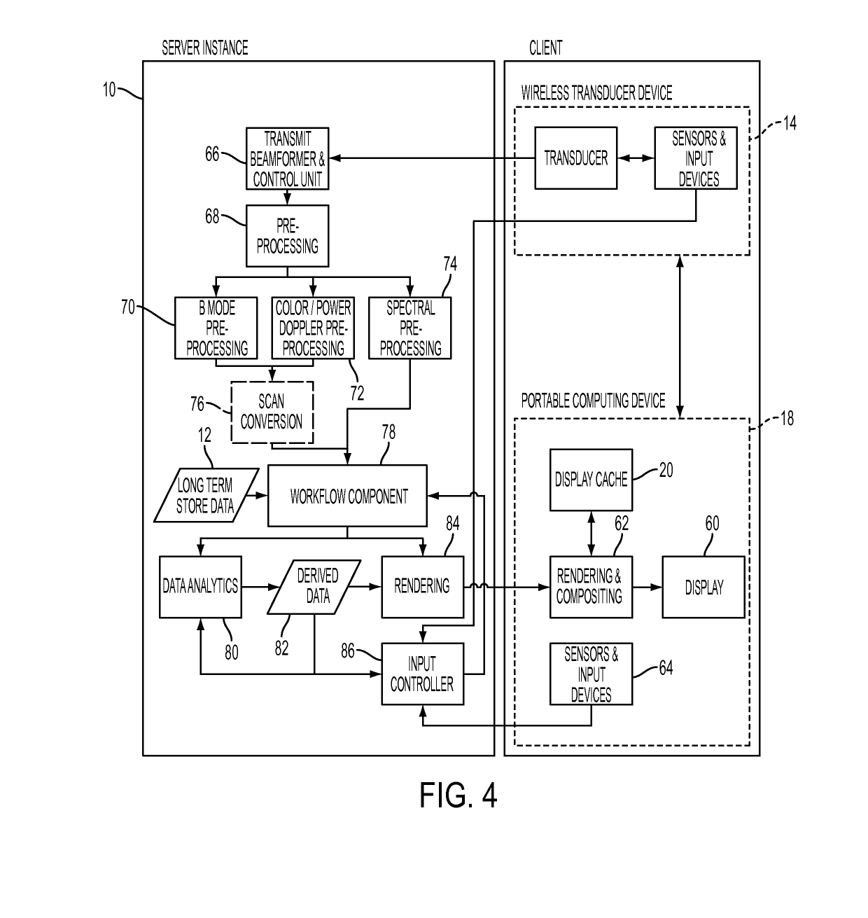

FIG. 4 shows one possible distribution for a given instance of ultrasound processing (given pair of the probe 14 and display 18). Each client is paired with a server instance. The server instance provides the processing, workflow and task control, rendering, and input control. As an alternative embodiment, depending on the capabilities of the client, the rendering may be done entirely by the server instance, entirely on the client, or rendered partially on each side and composited at the client. The transducer probe 14 may communicate states regarding the acquisition to the display 18 directly, or communicated through the server input controller 86.

The transducer probe 14, with the transducer array 15, generates channel data. Sensors or input devices 17 on the transducer probe 14 may be used to control the scan, activate the scan, control the image generation process on the server 10, and/or as feedback to determine resource sharing allocations.

The server 10 receives channel data from the array 15 of the transducer probe 14. The processor or processors of the server 10 perform actions to generate an image or sequence of images represented by the functions shown in the server instance in FIG. 4. The beamformer 66 controls the transmit beamformation of the transducer probe 14 and performs receive beamformation from the channel data.

The beamformed data is used for creating one or more images. Any preprocessing 68 is provided, such as time gain adjustment, filtering for harmonic information, phase adjustment, or line interpolation. Any mode of detection and corresponding scanning may be provided, such as B-mode 70, color, flow or Doppler estimation 72 (e.g., power, velocity, and/or variance), and spectral Doppler 74. The scan converter 76 converts the ultrasound data in the acquisition format to a format for the display 18. Other functions may be provided, such as filtering, mapping, and compositing. For two-dimensional imaging, the scan converter 76 or detectors output the image or images. For three-dimensional imaging, the rendering 84 renders the image (3D) or sequence of images (4D) from ultrasound data representing a volume rather than a plane.

The images are created as separate frames of data. Alternatively or additionally, the images are created with tiles. The image is divided into parts, each representing a different region of the image. The images may be stored in the memory 12. Other information may be stored in the memory 12, such as patient data for the data analytics 80.

The workflow component 78 manages the image processing of a given server instance. Additional information from the data analytics 80 (e.g., patient information) may be gathered and data derived 82 to be provided with the image or images. The workflow component 78, in conjunction with the input controller 86, controls the type of imaging, the image processing, data analytics 80, and transmission of the images to the display 18.

The input controller 86 receives user input, such as from the transducer probe 14 (inputs 17) and/or the display 18 (inputs 64). The input controller 86 is a processor acting as a change analyzer. The user input is used to control operation of the workflow component 78, image processing, and/or the transducer probe 14 (e.g., control the scan format and type of scanning). For example, the input controller 86 receives input from the display 18 for B-mode imaging. The input controller 86 causes the transducer probe 14 to perform a B-mode scan and causes the beamformer 66, pre-processing 68, B-mode processing 70, and scan conversion 76 to operate for creating a B-mode image from the channel data.

Acquired images, stored images, associated metadata, and/or client sensor data may be analyzed to determine change of scanning, processing, and/or display. For example, gyroscopes, accelerometers, light sensors or other sensors on the transducer probe or tablet display provide sensor information. The input controller 86 associates the received input with changes, such as sensing movement to activate or turn on the tablet display and sensing contact to activate the transducer probe or sensing movement or orientation to re-render an image from a different view direction.

The server may derive information from the acquired or stored images and/or associated metadata. For example, the analysis component may produce derived data that can be rendered and displayed with the images as text, overlays or shapes (e.g., locating an edge and overlap a graphic for the edge). As another example, the analysis component may utilize the client sensor data for analysis (e.g., identifying an organ being scanned based, in part, on orientation of the transducer). In another example, the analysis component may utilize other related image or non-image data associated with the image (e.g., using a previously acquired image of the same patient or from a different imaging modality in calculating a quantity, such as change in volume of a tumor).

The workflow component 78 causes the server 10 to transmit the images to the display 18. Where caching and/or tiling are provided, the workflow component 78 determines whether there is a change in each tile. Using communications, the number and/or identity of cached images and/or tiles on the display is determined. Alternatively, a standard or pre-determined cache is provided, and the server 10 knows the pre-determined caching. Past images and/or tiles in the memory 12 may be used to identify change. The workflow component 78 causes transmission of images and/or tiles that have been changed and avoids transmission of images and/or tiles already stored in the cache of the display 18. If the image or tile does not change over time, then the image or tile are not re-transmitted as long as the previous image or tile is available to the display 18.

The display 18 includes rendering and compositing 62. Any further image creation to be performed by the display 18 is provided. The compositing may be of different layers together (e.g., B-mode, Doppler mode, and graphic overlay). The compositing may be assembling tiles from caching 20 and/or from the server 10 into one or more images. The assembled image or images are output on a display device 60 of the display 18.

FIG. 5 shows one embodiment of a method for supporting multiple users with an ultrasound server. The method is implemented using the server 10 of FIG. 1, FIG. 2, FIG. 3, or another server. The server may interact with the transducer probe 14, display 18 or other devices to perform the acts. The acts are performed in the order shown, but other orders may be used. The acts are for serving multiple users in ultrasound imaging.

In act 22, communications between the server and the clients (e.g., transducer probes and displays) occurs. Data is transmitted from the transducer probe to the server, and image information is transmitted from the server to the display. User control, scan control, display control and/or other control information may be communicated between the components.

The communication is wireless. The communications are established as needed, upon power-up or startup of the transducer probe and/or display, or upon user initiation. Any protocol for establishing the wireless communications or networked communications may be used. The server establishes or manages the communications. Alternatively, the transducer probe and/or display establish or manage the communications.

The transmissions are for paired or linked devices, such that images created from scans of a given transducer probe are provided to the corresponding display. Since the same server may provide image processing for different scans, the pairings or communications linkages are unique. Using port, coding, frequency, addressing, keys, or other information, communications for each given pairing may be distinguished from other pairings. For example, spread spectrum transmissions are used using spreading codes unique to each pairing. The ultrasound imaging operations of different groupings of transducer probes and displays are maintained separate for communications.

In act 24, the server operates multiple instances of an ultrasound server. The server is a local server, such as within wireless communications range of the transducer probes and/or displays. For example, the server operates instances for scanning patients in different rooms or at different beds in a hospital, clinic, floor, department (e.g., emergency room), building, or area. For any active pairing or for any transducer probe being used to scan a patient, the server creates an instance of the ultrasound image processing application. The same program is initiated and operates separately for each transducer probe and/or display being used. Separate instances of an image processing and control system are operated by the same server. For example, an instance operates for a given pair of transducer probe and display. Other instances operate with other pairs of transducer probes and displays.

In one embodiment, the separate instances of the ultrasound system are paired with separate instances of an operating system. A virtual machine is created on the server for each transducer probe. The multiple instances operate as multiple virtual instances. A scalable design is realized with each software server instance virtualized within its own software virtual instance with its own operating system instance. Virtualized design is scalable to any arbitrary number of clients. Adding and removing support does not disturb the core design. Hardware resources such as CPUs and GPUs are accessed and shared through the virtualization layer. The virtualized design may incur more redundant resource usage by the extra operating system instances, which require more server hardware and more restrictions within virtualization technology versioning and support. In an alternative embodiment, virtualization is not used, instead running separate instances 90 as instantiated ones of the same program using a common operating system.

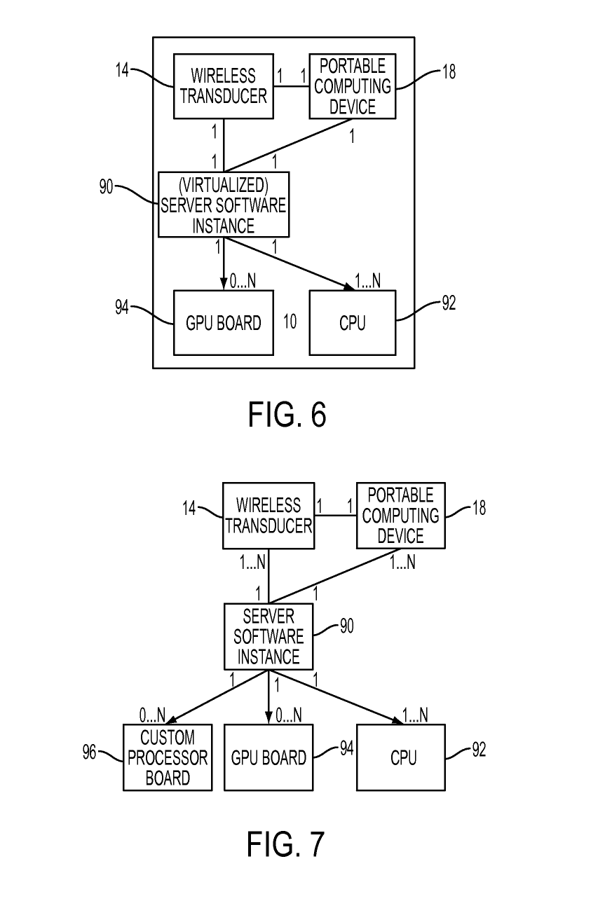

Multi-user support is achieved where one software server instance is paired with one client, so that multiple server instances run on one or more server systems to support multiple clients. FIG. 6 shows the instance relationship between the server, transducer, portable computing device and possible hardware resources (e.g., GPU 94 and CPU 92). The one instance 90 on the server communicates with one wireless transducer 14 and portable computing device (e.g., display 18). Since the server is running multiple such instances, the management of the shared resources (e.g., CPU 92, GPU 94, memory 12, interfaces, ports, and/or communications) is handled, at least partially, by the operating system of the server or other supervisor program. Each server instance queries for and acquires its own utilized resource on start up or dynamically based on utilization.

FIG. 7 shows an alternative embodiment. A single server instance connects with and image processes for multiple clients. The main difference in instance relation is that one server 10 and corresponding instance 90 is aware of and manages multiple clients (e.g., transducers 14 and displays 18) and potentially explicitly assigns processing hardware, such as the GPU 94, CPU 92, memory budget, communication ports, temporary storage for each client, and use of the custom board 96. The custom processor board 96, such as a receive beamformer or scan converter, may not operate in a shared manner with virtualization. Virtualization may cause conflicts for the custom resource not caused by mere time sharing or scheduling.

In either virtualized or not virtualized server design, the physical computing resources, such as CPU 92, GPU 94, memory 12, connection ports, and/or transmission bandwidth, are shared amongst the multiple client connections and are optimally managed by the server during operation. The shared ultrasound server resources are dynamically assigned based on idle/active status of the client side acquisition or review workflow.

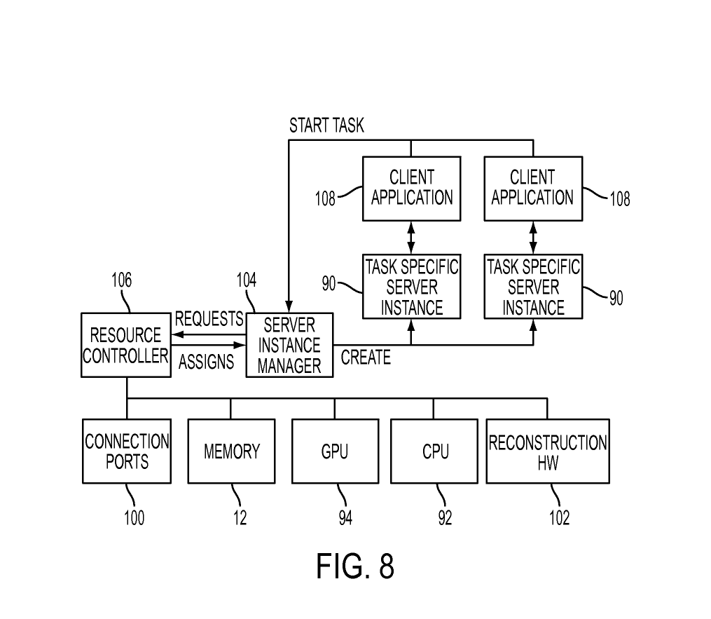

FIG. 8 shows one embodiment of connection and resource management. A possible control flow for server assignment of shared computing and communication resource to multiple clients is shown. Other flows may be provided. A server instance manager 104 is responsible for detecting any client initiation requests, shown as start task, from the client applications 108 (e.g., from the transducer probe and/or display). Based on the type of request, the server instance manager 104 requests the resource controller 106 to assign the necessary resource (e.g., connection ports 100, memory 12, GPU 94, CPU 92, and reconstruction hardware 102) to the server instance 90 for the client. Different clinical tasks and types of image acquisitions (e.g., type of scanning—B-mode, Doppler, spectral, or combinations thereof) may require different amounts of computing resources. A task mapper (e.g., processor with a look-up table) dynamically maps task requirements to required resource assignment to allow for flexibility and scalability of the server to new tasks and new probes. The resource controller 106 identifies minimums for each resource from the mapping based on the user input information from the client. Once the required task resource is identified, the resource controller 106 may search for the available connection channels/ports 100, hardware resources such as GPU 94, CPU 92, memory 12, or others and assign the server instance 90/client 108 pair to the respective resource.

To avoid one server instance 90/client 108 being assigned and hanging on to specific CPU 92, GPU 94, communication ports 100, transmission bandwidth, or memory 12 budgets where the resources are not later used, each server instance 90 may relinquish its claimed resource to the resource controller 106 to re-assign the resource to other client connections based on actual usage, needs, or changes in expected imaging.

The idle resource may be detected by one or in combinations of different approaches. A prolonged lack of motion activities at the transducer may be detected. If the workflow task is in the image acquisition state and the available gyroscope, inertia, accelerometer, light sensors, or ultrasound scan data has not detected any motion activities, images may not be actively being acquired. When the sonographer is imaging, the transducer probe is typically moved, at least within a one-five minute time frame. In another approach, cameras and video images may be used to detect a presence of operators or patients through explicit image or video-based human detection algorithms. Alternatively, change in the image over time in a camera image may indicate continued use. In yet another approach, signal analysis of ultrasound echo signals indicates whether the transducer is in contact with the patient skin for scanning or is not being used to scan. In other approaches, conductivity, capacitance, variance, inductance or other such electrical property is detected. The sensor is placed in the grip of the transducer probe or on the window for contacting the patient. The presence of contact by the operator or the patient indicates current use, and the absence of contact indicates no use. In yet another approach, a prolonged (e.g., 1-5 minutes) lack of change in the ultrasound signal, or detection of long delay echo signal indicates that the user does not have the transducer placed on the patient and may be used as an indicator that the user is not actively acquiring meaningful images. A prolonged lack of any type of user input from mouse, touch events, sensors at the portable computing device, especially during review mode, may also be used as an indicator of lack of activities, especially rendering activities to relinquish rendering and image transmission resources. A prolonged lack of rendered image input changes may also indicate that continued resource assignment for rendering is not required. In another example, the transducer is explicitly set to the freeze acquisition mode. Other changes in the type of imaging (e.g., changing from 3D imaging to 2D imaging) may indicate that resources may be reassigned. In yet another approach, the transducer or portable display device is set to suspend or off mode.

When a change in resource needs or utilization is detected, all or some of the resources may be reassigned or available for assignment to other instances or image processing. For continued use but by a different amount, the resource mapping may be repeated based on current settings. For a lack of any activity, all of the resources assigned to the instance may be recommitted or freed for use by other instances. If the server instance becomes active again or needs more resources due to another change, the server instance may request the resources.

During operation of the server instance, the server instance, transducer probe, and display interact. User input is used to establish the information to be exchanged, such as the display indicating the type of imaging to the server, and the server sending settings to the transducer probed for acquiring the desired data. The image processing performed by the server instance and the acquisition by the probe are controlled based on user inputs, such as from the display or the probe.

In act 26 of FIG. 5, image processing of data from the ultrasound probes is performed. Based on the assigned resources and instantiated server instances, the server creates images and provides the images for display. The control of the acquisition and receipt of data is also performed. For each of the image processing and control systems (e.g., server instances), the operation includes creating images by processing received data.

In one embodiment, the image processing includes beamforming channel data. The server receives channel data on the assigned port from the transducer probe. The channel data is delayed, phased, and/or apodized across the channels, and then combined.

The beamformed data is further processed, such as by detection, filtering, and gain adjustment, to generate one or more images. These images are transmitted to the display. In one embodiment, layering, caching, and/or tiling are provided. For example, only sub-sets of tiles associated with a change from a previous image are generated and transmitted. Subsequent images may be assembled from the sub-set of tiles for the image in combination with tiles from a previous image for unchanged regions. The display may assemble so the communications and processing bandwidth usage of the server is less by only operating on the sub-set of tiles associated with a change.

Simultaneous operation of multiple clients is a challenge to deliver responsive viewing and control of the images, especially within standard wireless device bandwidth. Besides the optimal assignment of computing and communication resources based on idle detection, acquisition and workflow dependent dynamic transmission selection techniques may be employed to address various types of viewing use cases. Such types of acquisitions may be determined by the type of transducer probe that is being used and/or the user input information. The acquisition type information may be determined by signals sent by the transducer, or by configurations at the portable computing device or at the server instance.

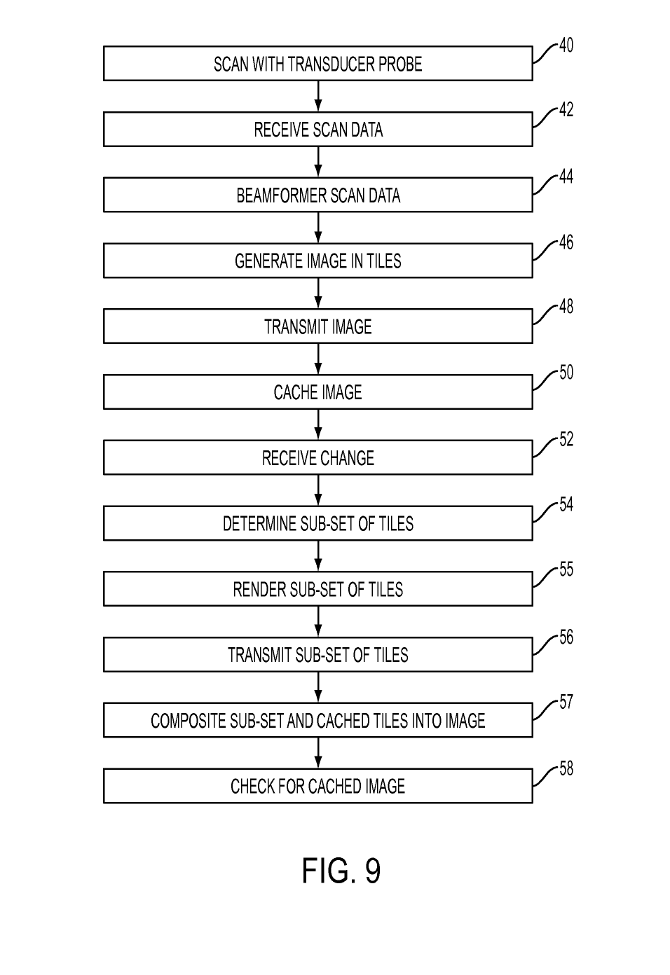

FIG. 9 shows a method using bandwidth reduction techniques. The techniques used depend on the type of images being acquired or reviewed. The method of FIG. 9 is implemented using the system of FIG. 1, FIG. 2, FIG. 3, or other system. The acts of FIG. 9 are performed by a local area server, a transducer probe, and/or a portable computing device (e.g., the display 18). Scan data is provided from the probe and to the local area server. The local area server receives data, processes data, and provides data to the portable computing device. The portable computing device displays the images. Control functions are managed by the local area server, but may be distributed or managed by other components.

Additional, different, or fewer acts may be provided. For example, caching acts 50 and 56 and/or tiling acts 48-54 are not provided. As another example, the receiving of a change 46 is not provided. Instead, the tiling and/or caching occur without a change being received from a user input.

The acts are performed in the order shown or a different order. For example, act 44 is performed prior to act 42, such as where the transducer probe performs receive beamforming. As another example, act 48 is performed after any of acts 46-54. Act 56 may be performed at any time.

In act 40, the transducer probe scans the patient. The user activates the probe. The activation causes the transducer probe to establish a communications link with the server. As the transducer probe scans the patient, channel data is output to the server over the communications link. From the user perspective, the user may merely power on the transducer probe and/or display to begin imaging. The probe is placed against the patient and scanning commenced. Received signals are digitized, buffered, and sent wirelessly to the server.

In act 42, the server receives the scan data. The ultrasound scan data is received wirelessly from the handheld transducer probe. The data is received by the established direct communications link, but may be received by routing through a network or over one or more relays.

The received scan data is channel data. Samples representing the received signals for each element are received. Alternatively, beamformed or partially beamformed data is received. The ultrasound data is of any mode, such as data from a B-mode, flow mode, M-mode, harmonic mode, spectral Doppler mode, or contrast agent mode scan.

In act 44, the channel data is beamformed by the server. Other processes may occur prior to beamforming, such as filtering. For beamforming, delays or phasing across data from different channels is applied. Apodization or amplitude weighting may also be applied. The data is combined to represent the echoes from different spatial locations along a scan line. The process is repeated over time for the scan line with dynamic focusing, and repeated for multiple scan lines. The server uses a processor and/or purpose built beamformer for beamforming.

In act 46, an ultrasound image is generated. Using detection appropriate for the imaging mode (e.g., B-mode, Doppler flow estimation, tissue Doppler estimation, spectral Doppler, harmonic, contrast agent, or other detection), the server determines (e.g., detects) values for imaging. The values may be filtered or other processes performed. Scan conversion may be provided.

In one embodiment, any ultrasound two-dimensional image processing may be provided. In other embodiments, the image represents a volume region of the patient, such as performing a three-dimensional rendering. Data representing a volume, such as representing a plurality of spaced apart planes in the patient, is rendered to a rendered image for display on a two-dimensional display. Any rendering may be used, such as projection (e.g., alpha blending, maximum intensity, or minimum intensity projection) or surface rendering. Lighting or other rendering effects may be provided.

A view direction is established by the server or input from the user. Clipping planes, segmentation, scale, or other user or processor controls of the data to be rendered may be provided. The user may later change some aspect of the rendering such that the same data is used to render another image.

In one embodiment, the image is generated in tiles. The 3D rendering or 2D images are created and divided into pieces. Alternatively, the image creation is performed separately for different parts of the image. Any size tiles may be used. The tiles have a same size (area) and shape (e.g., square), but may have different sizes and shapes, for a given image. For example, ultrasound images may be in a sector or Vector® format, so have a generally pie shape. The generation of the tiles may further leverage the knowledge about the scan region to omit detection of the changed tiles outside of the scan region.

In an alternative or additional embodiment, the image is layered. Different modes of imaging are provided separately as layers in the image. By combining the layers, a composite image is created, such as B-mode image with color flow information. Separate images are created for each layer of information. Separate tiling using the same or different tile regions may be provided for each layer.

In act 48, the ultrasound image is transmitted to the display. Where the image is formed of tiles, the tiles are sent. The tiles for the entire image are sent if it is the initial or first image for a scan sequence. If tiles of subsequent images are not different than cached tiles, these tiles that are the same are not sent. If image caching is used, the image may not be sent if the image is the same as a previous image, which is still cached by the display. Where tiling and/or caching are not used, each image in a sequence is sent. A single image may be sent for a freeze mode of imaging or rendering a given volume.

The transmission is wirelessly to the display. The transmission is addressed, coded, or encrypted for a specific display. Alternatively, a frequency or spread spectrum code is used to provide the image to a specific display instead of other displays. The display to which the image is transmitted is based on the source of the data. The image is transmitted for receipt by the display paired with the transducer probe used to acquire the data.

In one embodiment, the transmission bandwidth is limited using compression. Any lossless or lossy compression (e.g., Jpeg) may be used. For live two-dimensional imaging, the beamforming, preprocessing, rendering, or other image processing of the live transducer signal is performed as fast as possible to send the image to the client display. However, since much of the image quality is perceived from the animated aspects of the image stream, slightly lossy image compression may be acceptable without significant perceived loss. In this case, the processed images may be rendered or created and encoded as compressed image or video streams to the client portable device to display directly. In some cases, the decoding of the image or video stream may be further accelerated by the available portable device hardware either as dedicated hardware processor, or as generic programmable GPU or CPUs. Client side hardware decoding may consume less power than using software or CPU decoding.

In act 50, the image is cached. The client computing device, such as the display, caches the images. Any number of images may be cached, such as 10 or 100. The number may be based on memory resources or time instead of a specific number. Where the images are layered or tiled, the caching maintains the layering or tiling. Alternatively, the caching is for assembled or composited images. Prior to, after, or simultaneous with displaying the image, the image is stored in memory for later use. Where the image is formed from tiles and fewer than all regions are represented in the tiles, just the tiles received are cached. Alternatively, the image assembled from the current tiles and previous tiles is cached.

In act 52, a change is received. The server, probe, or display receives the change. The change is communicated to the server, probe, and/or display. Any change may be received. For example, scan settings are changed automatically by the server or based on user input. The image mode may be changed, such as based on user input. Where an on-going scan sequence is being performed, the change may be in the scan data, such as changes associated with anatomical movement. In one embodiment, the change is for volume rendering. The user, display processor, or server changes the lighting, viewing angle, clipping plane, scale, or other rendering characteristic. For example, the user changes the viewing angle interacting with the display of a current rendering on the display. The different view direction is communicated to the server. The server receives the change from the user input. The view direction may result in different rendering or selection of a different two-dimensional cross section. Change may alternatively or additionally be detected or received from a gyroscope, inertia sensor, accelerometer, light sensor, pressure sensor, heat sensor, or other sensor attached to or part of the transducer probe or the display device. The change is analyzed to determine scanning, processing, and/or display changes. These changes may lead to differences in resource allocation by the server, such as activation or change in rendering increasing bandwidth dedicated to a particular probe and display.

In act 54, the server determines a first sub-set of the tiles of the ultrasound image that are different due to the change and a second sub-set of the tiles that are not different. The subsequent image is generated and compared tile-by-tile to a previous image, such as the most recently generated image before the current image. The comparison may be a correlation, such as a sum of absolute differences. If there is no change or minimal change (e.g., below a threshold amount) between tiles of the different images, then the tile is determined to not have changed. If there is change, then the tile is to be transmitted to the display.

The determination is premised upon the display having cached the tiles that are not changing. The server may communicate with the display to determine whether the non-changing tiles are cached or to determine which tiles are cached. Alternatively, the caching procedure of the display is known to the server so that the server determines which tiles are cached or not without communicating with the display.

Determining by correlation or level of similarity may be processing intensive but result in use of less transmission bandwidth. To avoid or limit this processing, the determination may use a geometric proxy. For volume rendering, the region represented by ultrasound data or displayed segments of such data may be determined and modeled with a geometric shape. For example, a pyramid, cuboid, or other three-dimensional shape is sized to the scan region. When the view angle or other rendering characteristic changes, the shape is similarly changed (e.g., rotated for a change in view angle). Regions from the new view still outside or not intersecting this changed geometric proxy are determined as not changing and other regions are determined as changing. For regions within the proxy geometry, a comparison may be performed to determine whether the change resulting in any difference or these tiles are assumed to have changed.

In one embodiment, the determination is made as part of playback in volume rendering. For playback of already acquired images, the preprocessed images are stored on the server and rendered by the renderer on the server and/or client devices before sending to the client display. For static 3D images, the single volume may be manipulated by common operations such as zoom, pan, rotate, change of image intensity transfer function, clipping (e.g., limiting the field of view to a subset region of interest), altering segmentation, or other operations. Each operation may change the rendered image only in some parts as compared to the previous rendering. For example, clipping may leave some tiles of the scan region the same but alter others. For each new rendered image, the image tiles that are different compared to the previous corresponding tiles are rendered.

In another approach, layering is used. For use cases where the display image is composed of more than one layer, such as including data from different imaging modes and/or including a graphic overlay, each image layer may be individually composed of image tiles. The technique for static 3D imaging may be applied for each layer by the server.

A geometry layer may be provided for a graphic representing the scan volume or representing the scan volume relative to a patient. Such a geometric representation may be a wire frame model and small in size, such as for creating an icon. For geometry layers, since the geometry representation is usually small in transmission size, the geometry coordinates and primitives may be sent directly to the client and drawn by the client side rendering. Alternatively, the rendering is performed by the server as a single tile.

In act 55, the tiles of the selected sub-set (tiles associated with change) are rendered. Surface or projection rendering are performed for ultrasound data from a slab or portion of the volume corresponding to the tile. To save processing, the rendering is performed only for the tiles associated with change. For two-dimensional imaging, the image is created for the tiles or the tiles are extracted from the image.

In act 56, the rendered or created tiles are transmitted to the display. Tiles for regions not associated with change are not transmitted, saving bandwidth. For transmission, the tiles are packed and compressed as a single image. The tiles are aligned to avoid artifacts and packed into a single frame. The frame is compressed as if an entire image. The compressed image is transmitted to the display, which may decompress and un-pack the tiles.

In one embodiment, the rendered tiles are packed into a single image, then compressed, and transported to the client. For lossy compression, artifacts may result from such tiling since objects from discontinuous regions of the entire image are placed in adjacent tiles. To prevent this, the tiles are packed in a single vertical or horizontal alignment with a chosen tile size, color space, and compression sampling factor such that samples from adjacent tiles will not be combined in the compression space.

In act 57, the portable computing component receives the tiles. The tiles are decompressed, if compressed. The tiles are identified and used to composite with cached tiles to create an image. The tiles associated with change from the server are combined with tiles from one or more previous images from cache at the display to form a new image. The new tiles and/or image may be cached for later use.

In one embodiment, a display cache of tiles is held by the client such that the new tiles are drawn together with the unchanged tiles to compose the whole image. The display cache resides in memory, a file or as hardware textures or buffers.

For a special rendering effect to provide parallax perception, each client side layer and planar or 3D geometry may be rendered using a camera orientation and skew based on the local gyroscope sensor tilt of the portable display. Such rendering effect may provide a perception of the layer and geometry objects as it is viewed from a side view as the portable display is tilted physically.

In act 58 for full image caching in playback, the server checks for caching by querying the display. If images are cached by the display, the server does not create the images again and does not transmit the images to the display. Alternatively, the server knows of the caching without query. Only images, such as two-dimensional images, not cached are created and transmitted. This cache check is for full images rather than tiles. For a two-dimensional sequence playback, already played or provided images are used on the client side such that in case of rewind and replay, the client may invoke a request to the server only for the missing frames to be resent through a bi-directional request.

The server and client (e.g., transducer probe and/or display) perform various acts described herein using one or more processors. These processors are configured by instructions for supporting multi-user ultrasound. A non-transitory computer readable storage medium stores data representing instructions executable by the programmed processor. The instructions for implementing the processes, methods and/or techniques discussed herein are provided on non-transitory computer-readable storage media or memories, such as a cache, buffer, RAM, removable media, hard drive or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processors of the server and client components are a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device. The processor is a single device or multiple devices operating in serial, parallel, or separately. The processor may be a main processor of a computer, such as a laptop or desktop computer, or may be a processor for handling some tasks in a larger system, such as a graphics processing unit (GPU). The processor is configured by instructions, design, hardware, and/or software to perform the acts discussed herein.

While the invention has been described above by reference to various embodiments, it should be understood that many advantages and modifications can be made without departing from the scope of the invention. It is therefore intended that the foregoing detailed description be regarded as illustrative rather than limiting, and that it be understood that it is the following claims, including all equivalents, that are intended to define the spirit and the scope of this invention.

I (We) claim:

1. A method for supporting multiple users with an ultrasound server, the method comprising:
receiving, at a local area server, ultrasound scan data from a handheld transducer probe;
generating, by the local area server, an ultrasound image representing a rendering from the data, the ultrasound image comprising a plurality of tiles;
transmitting, from the local area server, the ultrasound image to a display;
receiving a change for the rendering;
determining a first sub-set of the tiles of the ultrasound image that are different due to the change and a second sub-set of the tiles that are not different;
rendering, by the local area server, the tiles of the first sub-set; and
transmitting, to the display, the rendered tiles of the first sub-set and not tiles of the second sub-set.

2. The method of claim 1 wherein receiving comprises receiving the ultrasound scan data as channel data from elements of the handheld transducer probe, and further comprising beamforming the ultrasound scan data.

3. The method of claim 1 wherein generating comprises volume rendering from a first view direction, wherein receiving the change comprises receiving a second view direction different than the first view direction, and wherein determining comprises determining the second sub-set as corresponding to tiles representing regions outside the scan region.

4. The method of claim 1 wherein transmitting the ultrasound image comprises transmitting the ultrasound image as the tiles, each tile representing a different two-dimensional region of the ultrasound image.

5. The method of claim 1 wherein determining the first sub-set comprises identifying regions of no change with a pre-computed three-dimensional geometry proxy.

6. The method of claim 1 wherein rendering the tiles comprises rendering only the first sub-set and not the second sub-set.

7. The method of claim 1 further comprising:
caching, by the display, the tiles of the ultrasound image;
compositing the rendered tiles of the first sub-set with cached tiles of the second sub-set.

8. The method of claim 1 wherein transmitting the rendered tiles comprises packing the rendered tiles of the first sub-set and compressing the packed, rendered tiles as a single image, and transmitting the compressed single image.

9. The method of claim 1 wherein transmitting the ultrasound image comprises transmitting the ultrasound image as a compressed image.

10. The method of claim 1 further comprising checking, by the local area server, for caching, by the display, of a sequence of two-dimensional images and transmitting only the two-dimensional images not cached.

11. The method of claim 1 wherein the ultrasound image comprises first and second layers, each layer corresponding to a different scan mode, and wherein determining the first sub-set of tiles comprises determining for the first layer, and further comprising determining tiling separately for the second layer.

12. The method of claim 1 further comprising operating multiple instances of an ultrasound system at the local area server, one of the multiple instances connected with the display and the handheld transducer probe, and other of the multiple instances connected with other displays and handheld transducer probes.

13. The method of claim 12 wherein operating the multiple instances comprises operating each of the multiple instances as a virtual instance, each virtual instance having a separate operating system instance.

14. The method of claim 1 wherein receiving the change comprises receiving accelerometer, gyroscope, inertia sensor, or light sensor input from the handheld transducer probe or the display.

15. The method of claim 1 wherein receiving comprises receiving three-dimensional data from the handheld transducer probe, wherein generating comprises generating the ultrasound image representing a volume rendering from the three-dimensional data, and wherein receiving the change comprises receiving the change for the volume rendering.

16. The method of claim 1 wherein receiving comprise receiving two-dimensional data from the handheld transducer probe, wherein generating comprises generating the ultrasound image representing a two-dimensional cross-sectional rendering from the ultrasound data, and wherein receiving the change comprises receiving the change as a location, orientation, or location and orientation of the two-dimensional cross-sectional rendering.

17. In a non-transitory computer readable storage medium having stored therein data representing instructions executable by a programmed processor for supporting multiple users with an ultrasound server, the storage medium comprising instructions for:
communicating, wirelessly, with multiple ultrasound transducer probes;
operating a separate instance of an image processing and control system for each of the ultrasound transducer probes; and
image processing, for each of the image processing and control systems as part of the operating, data from the ultrasound transducer probes.

18. The non-transitory computer readable storage medium of claim 17 wherein operating comprises operating the separate instances in separate respective virtual machines.

19. The non-transitory computer readable storage medium of claim 17 wherein operating comprises sharing hardware resources of a server among the separate instances based on idle and active status of the transducer probes.

20. The non-transitory computer readable storage medium of claim 17 wherein image processing comprise generating images with tiles and updating only sub-sets of the tiles for subsequent images.

21. The non-transitory computer readable storage medium of claim 17 wherein image processing comprises beamforming channel data from the ultrasound transducer probes, wherein communicating further comprises transmitting images to tablet displays separate from and paired with the ultrasound transducer probes, and wherein operating comprises controlling the image processing and the ultrasound transducer probes based on user inputs from the tablet displays.

22. A system for supporting multiple users with ultrasound processing, the system comprising:
a plurality of ultrasound probes configured to scan patients and wirelessly output channel data;
a plurality of tablet displays paired with the ultrasound probes, each of the tablet displays configured to operate as a user input for control of the paired ultrasound probe;
a server configured to receive the channel data from the ultrasound probes, beamform the channel data, create images from the beamformed channel data as a function of the user input from the tablet displays, and transmit the images to the tablet displays, and configured to control the ultrasound probes as a function of the user input.

23. The system of claim 22 wherein the tablet displays each comprise a cache configured to store tiles of displayed images, the server configured to create the images in tiles and avoid transmitting tiles for parts of the images that do not change over time, the tablet displays configured to create displays assembled from the tiles in cache from the displayed images and from tiles updated by the server, the tiles being specific to type of scan mode.

24. The system of claim 22 wherein the ultrasound probes, tablet displays, or ultrasound probes and tablet displays comprise one or more input sensors comprising touch sensors, video cameras, gyroscopes, accelerometers, inertia sensors, or combinations thereof, and wherein the server is configured to determine resource allocation as a function of input from the input sensors.

25. The system of claim 22 wherein the server is configured to produce data derived from images and associated metadata.
